(19) United States
(12) Patent Application Publication
(10) Pub. No.: US 2026/0144490 A1
(43) Pub. Date: May 28, 2026

(54) DEVICES AND METHODS FOR A NON-INVASIVE HAND-TO-HAND ELECTROCARDIOGRAM TEST DURING PACED BREATHING TO MEASURE, ANALYZE AND MONITOR VAGUS NERVE ORIGINATED CARDIAC- AND RESPIRATORY EFFECTS WHICH CAN BE USED FOR HEALTH MONITORING, MEDICAL DIAGNOSTICS AND PERSONALIZATION OF HEALTH CARE

(71) Applicant: Vagus Health Ltd., Cambridge (GB)

(72) Inventor: Gustaf Kranck, Cambridge (GB)

(21) Appl. No.: 19/412,947

(22) Filed: Dec. 9, 2025

Related U.S. Application Data
(63) Continuation of application No. 17/502,212, filed on Oct. 15, 2021, now abandoned.
(60) Provisional application No. 63/091,952, filed on Oct. 15, 2020.

(51) Int. Cl. A61B 5/00; A61B 5/024; A61B 5/08; A61B 5/11; A61B 5/28; A61B 5/346

(52) U.S. Cl. CPC A61B 5/6824; A61B 5/024; A61B 5/0816; A61B 5/11; A61B 5/28; A61B 5/346; A61B 5/6826; A61B 5/742

(57) ABSTRACT

A wrist or finger mounted wearable device and method for determining a state or activity level of the autonomic nervous system, such as the respiratory rate or activity of the vagus nerve. The wearable device having at least a pair of electrocardiograms (ECG) measurement electrode pads configured to perform hand-to-hand ECG measurements. The device or method including monitoring of the pair of ECG measurement pads at a first time period to arrive at a first reading of hand-to-hand ECG, and at a second time period to arrive at a second reading of hand-to-hand ECG. The device or method analyzing the first and second readings of ECG to arrive at various indications of the state of a user.

### BACKGROUND

The vagus nerve is the longest and most complex of the 12 pairs of cranial nerves. It transmits information to and from the brain to organs in the body such as the gut and heart. The vagus nerve is the main component of the parasympathetic nervous system which is also known as the ‘rest and digest’ system. The ‘opposite’ of this system is the sympathetic nervous system which is known as the ‘fight and flight’ system. It is activated when the body is in stress such as during disease progress.

The autonomic nervous system is the main nervous system involved in the body's stress response. The vagus nerve is a very important part of the autonomic nervous system which enables, for instance, relaxation. The vagus nerve is also the most important nerve for immune system responses and it is considered to be very important in the treatment of autoimmune diseases, depression, anxiety and other diseases that require lowering or raising the immune system responses.

Recent advances in immunology reveal a significant pathogenic role for inflammation in the development and progression of inflammations in cardiac- or respiratory systems. The worldwide onset of COVID-19 has revealed a lack of digital health monitoring and early detection systems of such diseases.

### SUMMARY OF THE INVENTION

The invention is defined by the features of the independent claims. Some specific embodiments are defined in the dependent claims.

According to a first aspect of the present invention, there is provided A wrist or finger mounted wearable device for determining a state or activity level of the autonomic nervous system, such as the respiratory rate or activity of the vagus nerve, the wearable device comprising: at least a pair of electrocardiograms (ECG) measurement electrode pads configured to perform hand-to-hand ECG measurements, at least one processing core and at least one memory including computer program code, the at least one memory and the computer program code being configured to, with the at least one processing core, cause the wearable device to: monitor the pair of ECG measurement pads during a test period while providing for instructions to perform controlled breathing, to arrive at a reading of hand-to-hand ECG, analyze the reading of hand-to-hand ECG to arrive at indications of at least the following: respiratory rate, and pulse.

According to a second aspect of the present invention, there is provided a method of determining a state of health for an individual by monitoring the breathing of an individual via hand-to-hand electrocardiograph, the method comprising the steps of: a first phase of recording the heartbeat of the individual via hand-to-hand electrocardiograph (ECG) during normal breathing, a second phase of recording the heartbeat of the individual via hand-to-hand ECG during paced or controlled breathing, analyzing from second recordings to arrive at an indication of a state of function of the autonomic nervous system of the individual, wherein the first phase lasts at least 30 seconds and the second phase lasts at least 30 seconds, preferably 60 seconds.

### BRIEF DESCRIPTION OF THE DRAWINGS

FIG. 1 illustrates a smartwatch in accordance with at least some embodiments of the present invention as employed to perform a hand-to-hand ECG test;

FIGS. 2A-2C show a smartwatch according to certain embodiments of the present invention which prompts a user during performance of a hand-to-hand ECG test via a display of the smartwatch;

FIG. 3 is an example apparatus capable of supporting at least some embodiments of the present invention;

FIG. 4 illustrates an example user interface capable of supporting at least some embodiments of the present invention;

FIG. 5 shows an outcome matrix demonstrating how the system is establishing a clinical diagnosis from Respiratory Sinus Smoothness (RSS index 0-100 on y-axis) and Cardiac Sinus Smoothness (CSS index as 0-100 on x-axis).

FIGS. 6-12 shows an example breathing and heart rate graph derived from readings during a test according to certain embodiments of the present invention;

FIG. 13 shows a QRST profile of an ECG recording;

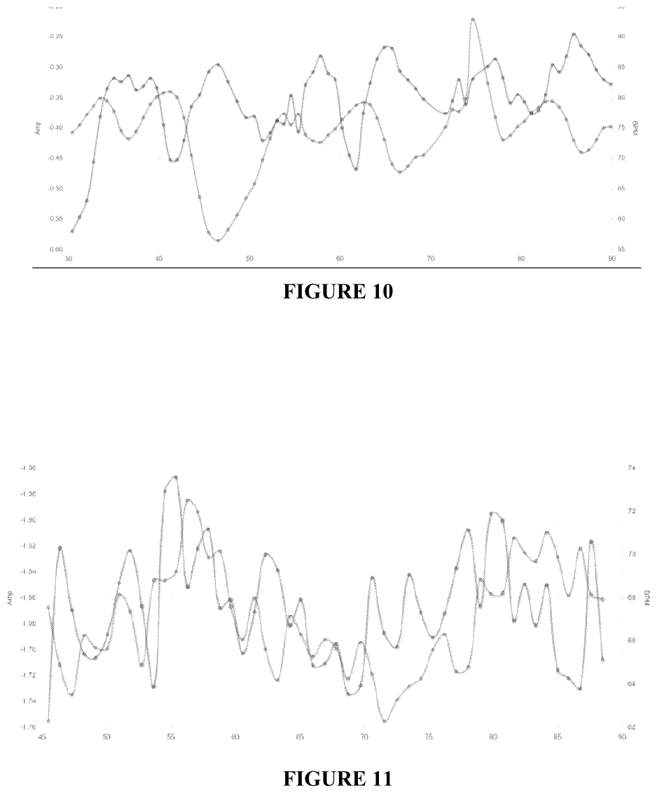

FIG. 14 shows the vagus nerve and connected systems;

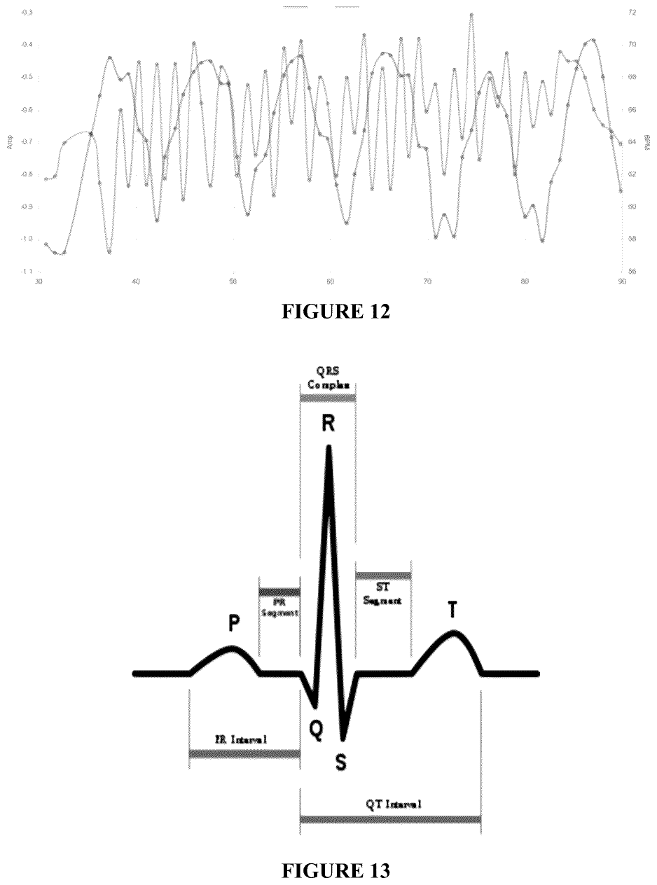

FIG. 15 shows baseline electrocardiograms for selected days within a 49 day progress of a disease;

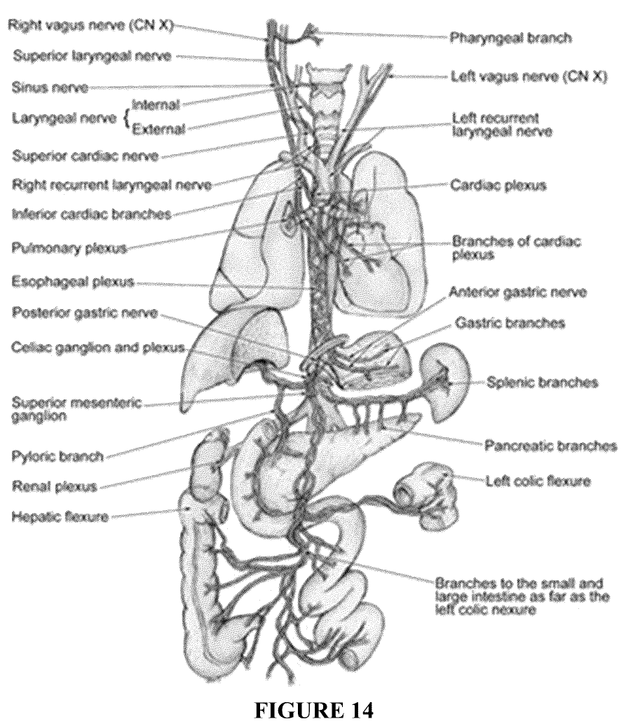

FIG. 16 shows mean QRS voltage and heart rate versus days from inoculation; and

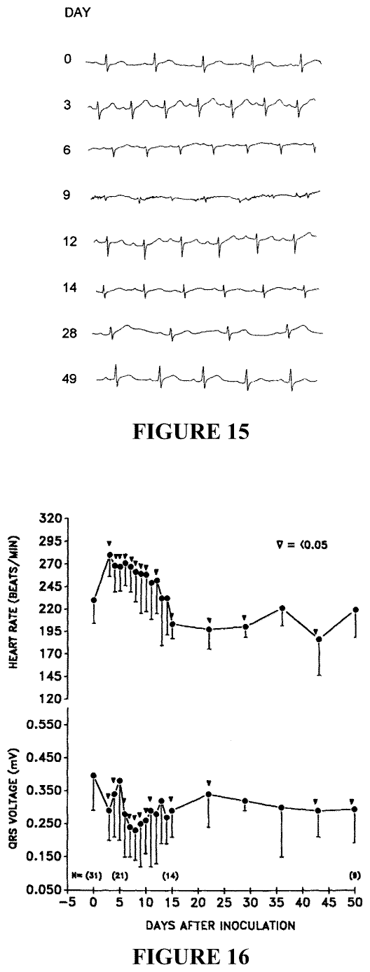

### EMBODIMENTS

### Definitions

In the present context, the term ‘vagal’ is used as ‘relating to the vagus nerve’.

Vagal tone refers to the level of activity and health of the vagus nerve.

Heart rate variability (HRV) provides for a method to evaluate vagal tone or vagus nerve activity. However, HRV has a major statistically originating flaw when used in short term measurements. Since the heart beats only on average once per second during a resting heart rate—the statistical accuracy when measuring only 20 or 30 heartbeats (25 second test), is on average low. However, methods and devices according to embodiments of the present invention monitor amplitude changes and electrocardiogram's (ECG) QRST-peaks and internal patterns in order to arrive at a metric which can reveal consistent early indicators for the onset of disease. Further, the amplitude changes and electrocardiogram's (ECG) QRST-peaks and internal patterns act as consistent early indicators for the onset of disease.

Certain embodiments of the present invention provide for a Vagus ECG Test as a novel standardized test protocol enabling disease, immune system and vagus nerve measurement and assessment. In recording and analysis of more than 25 000 hand-to-hand ECGs, embodiments of the present invention have been proven to detect changes in the cardiac-respiratory synchronization (RSAsync) patterns when the users have become sick or have mentally derived issues which affect the vagus nerve. These changes in RSAsync can be used for early indicators of disease, infection and continuous monitoring of disease. Such changes can also be used to monitor the effects of medication.

In China the Coronavirus prevention during first months were focusing on mitigating the spread of the virus by preventing people from travelling and meeting in large crowds. Healthcare professionals and officials in China and around the world are currently conducting large scale screenings in public spaces, public transport and for instance when crossing borders. There currently exists only three non-invasive traditional risk screening methods for Coronavirus: 1) body temperature 2) outwards signs such as coughing 3) self-reporting of ‘feeling sick’. All of these screening methods are detecting the disease only after the person has become sick. When the screening- or healthcare professionals evaluate the risk of infection to be high, for example using embodiments of the present invention, the person can be quarantined, CT-scans are done for signs of pneumonia and a blood test is collected for PCR detection.

RSAsync according to certain embodiments of the present invention is a measurement of cardiac/respiratory synchronization. RSAsync patterns can provide for reliable and consistent indication when users have become sick or have mentally derived issues which affect the vagus nerve. Heart rate and breathing are generally considered to be synchronized by the oxygenation effect, whereby heart rate increases at inspiration and decreases at expiration. It has been shown that when the immune or nervous systems in general are disrupted, this synchronization is altered or disappears. These changes in RSAsync can be used for early indicators of disease, infection and continuous monitoring of disease- and for instance medication effects.

When studying the respiratory and RSAsync effects in hand-to-hand ECG during a controlled breathing test period, a systematic correlation between the user's cardiac health and the smoothness of the pulse curve has been proven. Similarly, there is a systematic correlation between the smoothness of the respiratory data curve and the user's reported respiratory health problems. A mathematical model to establish this smoothness is provided per certain embodiments of the invention and may be references as Cardiac Sinus Smoothness (CSS) and Respiratory Sinus Smoothness (RSS).

RSAsync, CSS and RSS can reliably be monitored and measured with the system presented herein comprising at least a ‘hand-to-hand ECG watch or ring which implements the standardized Vagus ECG Test consisting of at least a period of normal resting ECG measurement followed by a controlled breathing phase where the user is instructed how to conduct regular inhales/exhales. For example, regular inhales/exhales each lasting at least 5 seconds. Within at least some embodiments a cloud based analytics system is employed which uses big data processing.

According to embodiments of the present invention, the electrical potential difference is measured directly between hands as the amplitude of voltage measurements changes when the person inhales and exhales. This was first studied, recognized and documented by Dr. Brody in 1950s. Hence the commonly used ‘Brody effect’ for ECG voltage amplitude changes due to breathing. The reason for these voltage amplitude changes are as follows. During inspiration, there is diaphragmatic contraction in the heart that, because the pericardium is attached to the central tendon of the diaphragm, causes caudal and rotational movement of the heart (and vice versa). This rotational movement in term cause changes in conduction especially in the direction of a VR and a VL electrodes in standard 12-lead ECG measurement. This amplitude change is clearly visible as changes in the R and S amplitude in the hand-to-hand ECG and it is the reason why breathing can be calculated from ECG measurements.

Embodiments of the present invention enable a new method and test protocol to calculate the synchronization discussed above and its implications with the use of hand-to-hand ECG-derived respiration (EDR) periods. The diagnosis provides explanations for the surprisingly common alternations to this synchronization (coherence) of breathing and heart rate sinus arrythmia.

Devices and systems according to embodiments of the present invention can establish a novel and very informative parameter which is invented to be called Respiratory Sinus Arrhythmia Synchronization (RSAsync) as the measurement of the above described cardiac/respiratory synchronization. RSAsync may be used within certain embodiments of the present invention to describe and quantify how well the vagus nerve is able to provide optimal oxygenation and blood pressure to the body during breathing and especially during the tests as discussed herein such as the Vagus Test of Vagus ECG test. At least some embodiments employ hand-to-hand ECG in determining an indication of vagal tone, vagal homeostasis and immune system activity levels.

Certain embodiments of the present invention provide for calculation of a novel Respiratory Sinus Smoothness (RSS) value from Vagus ECG Test data. This value expresses how well the respiratory curve is corresponding to an optimized sinus curve most fitting the respiratory data. This calculated smoothness is compared to this optimal sinus curve and may be described as an index between 0-100 where 100 represents an very good smoothness-correlation to an optimal sinus curve.

This RSS value represents an important diagnostics value whereby the Vagus ECG Test analysis makes the diagnostics whether or not the test person is likely to suffer from respiratory illness or inflammations.

Some embodiments of the present invention provide for a novel Cardiac Sinus Smoothness (CSS) value calculated from the Vagus ECG Test data, expressing how well the cardiac curve is corresponding to an optimized sinus curve most fitting the cardiac pulse data. The smoothness of the factual curve, compared to the calculated optimal sinus curve, is described as a CSS index between 0-100 where 100 represents a very good smoothness-correlation of the cardiac curve to an optimal sinus curve.

When this CSS index is low, the test person might have cardiac health issues. This CSS value represents the most important classification whereby the analysis makes the diagnostics about whether or not the test person is likely to suffer from cardiac illness or inflammations.

Embodiments of the present invention provide for a system that enables the measurement of the test persons breathing capability and relative strength during conscious controlled breathing compared to base breathing. As an integral part of this system is establishing this capability and quantify it as a ‘Vagus Breathing Index’ (VBI).

In order to obtain a good measurement of the heart rate changes as compared to breathing activity, it is best achieved by using so-called controlled breathing whereby the user consciously inhales and exhales at longer periods than during normal (not consciously influenced) breathing. In order to reach optimal dynamic blood pressure and oxygenation, the heart rate should normally start increasing and decreasing before the end of each breath-cycle change.

A Vagus ECG Test according to certain embodiments of the present invention enables measurement of both base- and controlled breathing and comprises 2 phases. First the user is recording the hand-to-hand ECG with normal base breathing for at least 30 seconds and then the user is consciously affecting breathing so that each inhale and exhale is lasting at least 5 seconds each. This controlled breathing test period should last at least 1 minute.

One important function of the vagus nerve is that it is synchronizing the heart beats to breathing in order to optimise oxygen delivery to body tissues. This function is more active when the body is relaxed. When the body is stressed from mental issues or disease, this heart/breathing synchronization is altered.

The heart rate variability (HRV) during controlled breathing test in this system are compared to the HRV during base breathing phase directly prior to controlled breathing. The relation between these HRV values are part of the indication for the health of the autonomic nervous system (HR Vrel).

The here described method to test and calculate RSA, RSAsync, RSS, CSS, HR Vrel, HRmax, HRmin, HR and VBI as together indicators of an individual's autonomic nervous system state and health are novel applications and combination of data for diagnostic purposes.

The vagus nerve and general health of the user—with a sufficient degree of precision—can be characterized by combining two or more of the following values: RSA, RSAsync, RSS, CSS, Pulse, Breathing Index, HRV, HRV change during the Vagus ECG Test, Maximum and Minimum heart beat levels, change of heart activity when comparing base breathing to control breathing, activity levels, sleep, blood

Embodiments of the present invention overcome typical issues with short timespan HRV measurements and tests (<5 min), that they are generally unreliable, by combining some number of parameters, Respiratory Sinus Arrythmia (RSA), Cardiac/Respiratory Synchronization (RSAsync), heart rate variability (HRV), resting pulse and other bio-signal measurements from wearable devices—it is possible to make better and more comprehensive vagus nerve activity analysis than with traditional HRV analytics.

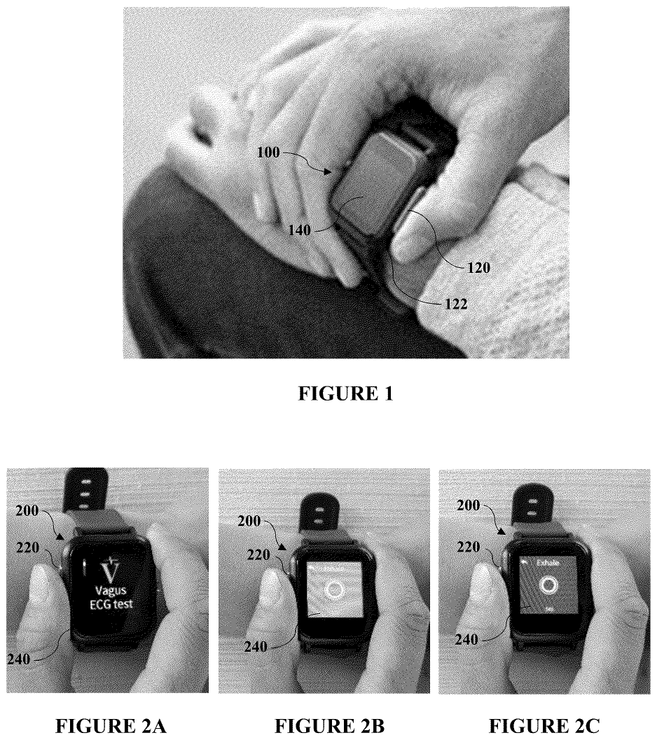

FIG. 1 illustrates a smartwatch 100 in accordance with at least some embodiments of the present invention as employed to perform a hand-to-hand ECG test. As shown, the wrist or finger mounted wearable device 100 for determining a state or activity level of the autonomic nervous system, such as the respiratory rate or activity of the vagus nerve is mounted on the wrist of the person and the other hand is brought into contact with a ECG pad 120. The other ECG pad 122 being mounted on the base of the watch. In least some embodiments of the present invention wireless forms of ECG measurement are employed in place of or in addition to the pads, for example, infrared ECG measurement. ECG pads as discussed herein may take a wide variety of forms and at least some embodiments at least partially employ wireless ECG measurement.

As per FIG. 1, the wearable device 100 comprises at least a pair of electrocardiograms (ECG) measurement electrode pads 120 and 122 configured to perform hand-to-hand ECG measurements. The device 100 also comprises at least one processing core and at least one memory including computer program code are comprised within the smartwatch of FIG. 1. The at least one memory and the computer program code being configured to, with the at least one processing core, cause the wearable device to: monitor the pair of ECG measurement pads 120,122 during a first test period to arrive at a first reading of hand-to-hand ECG, following the first test period, provide for instructions to the user to breathe in a normal fashion for a period prior to executing a second test period, monitor the pair of ECG measurement pads (120, 122) during the second test period, while providing instructions to perform controlled breathing, to arrive at a second reading of hand-to-hand ECG, analyze the first and second readings of ECG to arrive at indications of at least one of: a minimum, maximum and average pulse, a size of pulse changes, a smoothness of pulse changes, a respiratory rate, a depth of respiration, and a smoothness of respiration. In certain embodiments, a plurality, at least two, of the indications are calculated. In still other embodiments, every indication is derived.

Certain embodiments of the present invention may also be represented within FIG. 1 as follows. The wrist or finger mounted wearable device 100 for determining a state or activity level of the autonomic nervous system, such as the respiratory rate, heart rate or other below mentioned indications of the vagus nerve, the wearable device comprising: at least a pair of electrocardiograms (ECG) measurement electrode pads 120, 122 configured to perform hand-to-hand ECG measurements, at least one processing core and at least one memory including computer program code, the at least one memory and the computer program code being configured to, with the at least one processing core, cause the wearable device 100 to: monitor the pair of ECG measurement pads (120, 122) during a test period, while providing for instructions to perform controlled breathing, to arrive at a reading of hand-to-hand ECG, analyze the reading of hand-to-hand ECG to arrive at indications of at least the following: respiratory rate, and pulse.

At least some embodiments of the present invention provide for instructions to perform controlled breathing by sending signals to another device, for example a device with a display the user can read, such as a smartphone. In certain embodiments, instructions are provided for by displaying them directly on the wrist or finger mounted wearable device.

As also shown in FIG. 1, at least some embodiments of the present invention provide for a wrist or finger mounted wearable device further comprising a display 140. Such a display may be configured to provide prompts to a user, for example via text or graphics, such as animated graphics. As such, instructions may be provided to the user. In some embodiments, the device is further configured to provide the instructions to perform controlled breathing such that the user is prompted to breathe in a predetermine fashion, such as instructing the user to breathe at a particular rate, in particular to breathe at a frequency of 0.1 Hz.

Certain embodiments of the present invention provide for an indication of breathing or respiratory rate by measuring electrical potential difference directly between hands of a person. In such situations, the amplitude of voltage measurements change when the person inhales and exhales.

The reason for these voltage amplitude changes are as follows; during inspiration, there is diaphragmatic contraction in the heart which, because the pericardium is attached to the central tendon of the diaphragm, causes caudal and rotational movement of the heart and vice versa. This rotational movement in turn cause changes in conduction especially in the direction of aVR and a VL electrodes in standard 12-lead ECG measurement. This amplitude change is clearly visible as changes in the R and S amplitude in the hand-to-hand ECG and provides for indications of breathing in at least some embodiments of the present invention.

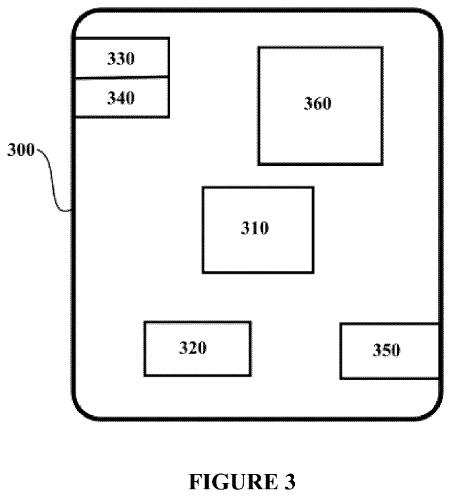

FIGS. 2A-2C show a smartwatch 200 according to certain embodiments of the present invention which prompts a user during performance of a hand-to-hand ECG test via a display 240 of the smartwatch while the user places their thumb on an ECG pad or sensor 220 of the smartwatch 200.

As shown in FIG. 2A the user is informed that a Vagus ECG test has been initiated and is waiting for instruction. The user is then prompted to breathe in a particular fashion by FIG. 2B instructing the user to inhale for a predetermined time and then to exhale for a predetermined time within FIG. 2C.

Within at least some embodiments of the present invention, the device is further configured to analyze indications of the respiratory pattern together with cardiac activity of the user derived from the second reading to arrive at an indication of the state or activity level of the vagus nerve.

In certain embodiments of the present invention, the device is further configured to compare the first readings and second readings to further analyze respiratory and cardiac patterns. In some embodiments, the first test period is at least 30 seconds and the second test period is at least 30 seconds but preferably at least 60 seconds. Within at least some embodiments the ECG recording is executed on the device only during the period when the user is breathing at a particular rate, this period lasting at least 30 seconds, but preferably at least 60 seconds.

Some embodiments of the present invention provide a device that is further configured to prompt a user to breathe normally during the first test period. In certain embodiments the device is configured to prompt a user in coordination with an external device, such as a mobile phone.

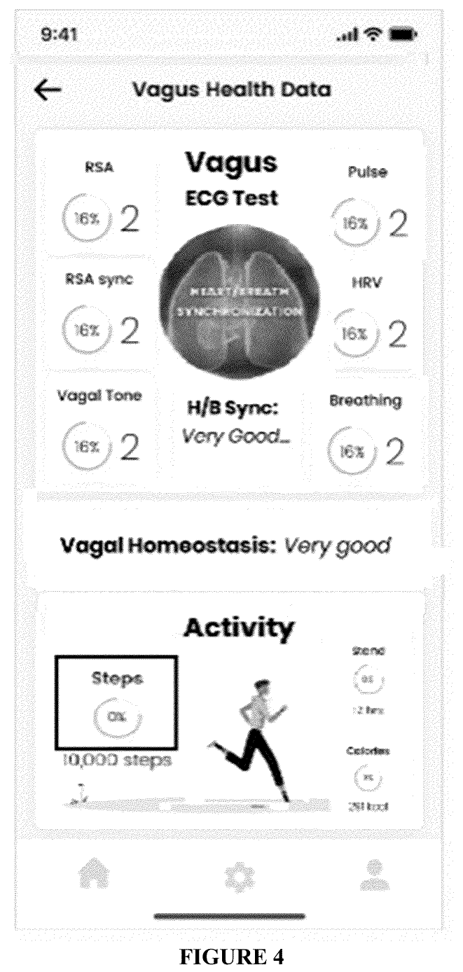

FIG. 3 illustrates an example apparatus capable of supporting at least some embodiments of the present invention. Illustrated is device 300, which may comprise, for example, a computer device such as controller 150 of FIG. 1. Comprised in device 300 is processor 310, which may comprise, for example, a single- or multi-core processor wherein a single-core processor comprises one processing core and a multi-core processor comprises more than one processing core. Processor 310 may comprise a Qualcomm Snapdragon 800 processor, for example. Processor 310 may comprise more than one processor. A processing core may comprise, for example, a Cortex-A8 processing core manufactured by Intel Corporation or a Brisbane processing core produced by Advanced Micro Devices Corporation. Processor 310 may comprise at least one application-specific integrated circuit, ASIC. Processor 310 may comprise at least one field-programmable gate array, FPGA. The aforementioned processor types are non-limiting examples, alternatively an Intel i7 processor, or another suitable type of processor, may be employed.

Device 300 may comprise memory 320. Memory 320 may comprise random-access memory and/or permanent memory. Memory 320 may comprise at least one RAM chip. Memory 320 may comprise magnetic, optical and/or holographic memory. Memory 320 may be at least in part accessible to processor 310. Memory 320 may be means for storing information. Memory 320 may comprise computer instructions that processor 310 is configured to execute. When computer instructions configured to cause processor 310 to perform certain actions are stored in memory 320, and device 300 overall is configured to run under the direction of processor 310 using computer instructions from memory 320, processor 310 and/or its at least one processing core may be considered to be configured to perform said certain actions.

Device 300 may comprise a transmitter 330. Device 300 may comprise a receiver 340. Transmitter 330 and receiver 340 may be configured to transmit and receive, respectively, information in accordance with systems, for example transmitter 330 may transmit information to a monitor for display to a user, and/or receiver 340 may receive input information concerning a location and/or orientation of a further device.

Device 300 may comprise a near-field communication, NFC, transceiver 350. NFC transceiver 350 may support at least one NFC technology, such as NFC, Bluetooth, Wibree or similar technologies.

Device 300 may comprise user interface, UI, 360. UI 360 may comprise at least one of a display, a keyboard and a touchscreen. A user may be able to operate device 300 via UI 360, for example to start or terminate execution of programs.

Processor 310 may be furnished with a transmitter arranged to output information from processor 310, via electric leads internal to device 300, to other devices comprised in device 300. Such a transmitter may comprise a serial bus transmitter arranged to, for example, output information via at least one electric lead to memory 320 for storage therein. Alternatively, to a serial bus, the transmitter may comprise a parallel bus transmitter. Likewise, processor 310 may comprise a receiver arranged to receive information in processor 310, via electrical leads internal to device 300, from other devices comprised in device 300. Such a receiver may comprise a serial bus receiver arranged to, for example, receive information via at least one electric lead from receiver 340 for processing in processor 310. Alternatively, to a serial bus, the receiver may comprise a parallel bus receiver.

Device 300 may comprise further devices not illustrated in FIG. 3. For example, where device 300 comprises a computer device, it may comprise at least one clock or auxiliary power unit, APU to provide battery power in case of mains power failure.

Processor 310, memory 320, transmitter 330, receiver 340, NFC transceiver 350 and/or UI 360 may be interconnected by electric leads internal to device 300 in a multitude of different ways. For example, each of the aforementioned devices may be separately connected to a master bus internal to device 300, to allow for the devices to exchange information. However, as the skilled person will appreciate, this is only one example and depending on the embodiment various ways of interconnecting at least two of the aforementioned devices may be selected without departing from the scope of the present invention.

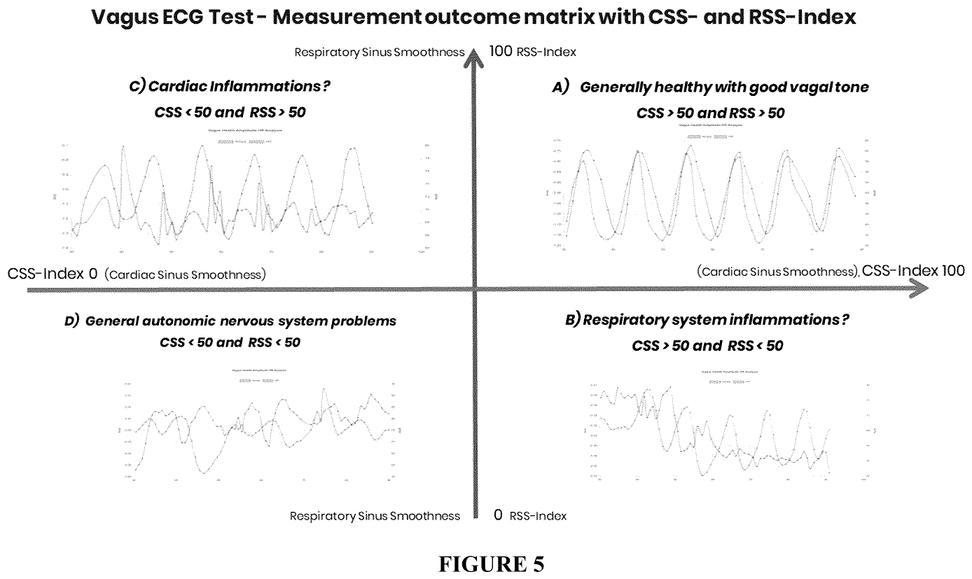

FIG. 4 illustrates an example user interface capable of supporting at least some embodiments of the present invention, displaying a user's health changes and disease risk assessments.

Certain embodiments of the present invention provide for a method of determining a state of health for an individual by monitoring the breathing of an individual via hand-to-hand electrocardiograph, the method comprising the steps of: a first phase of recording the heartbeat of the individual via hand-to-hand electrocardiograph (ECG) during normal breathing, a second phase of recording the heartbeat of the individual via hand-to-hand ECG during paced or controlled breathing, and analyzing from second recordings to arrive at an indication of a state of function of the autonomic nervous system of the individual, wherein the first phase lasts at least 30 seconds and the second phase lasts at least 30 seconds, preferably 60 seconds.

In some embodiments, a statistical analysis of the breathing data is performed in order to arrive at an index representative of state of health, such as a Breathing Index, Respiratory Sinus Arrythmia Synchronization, Respiratory Sinus Smoothness, Cardiac Sinus Smoothness and Vagus Health Index. In certain embodiments, the statistical analysis is at least one of: a percent variation and a percent of average analysis.

In certain embodiments the normal breathing data and controlled or conscious breathing data is compared in order to arrive at an index indicative of the state of health of the autonomous nervous system, such as a Vagus Beathing Index (VBI), Respiratory Sinus Arrythmia Synchronization (RSAsync) or Vagus Health Index (VHI).

At least some embodiments of the present invention derive breathing and cardiac curves from recordings during the paced breathing, the curves being to mathematically establish how the measured results conform to the closest possible optimal normal or asymmetric sinus curve and calculating the degree of error from these ideal sinus curves to establish Respiratory Sinus Smoothness (RSS) and Cardiac Sinus Smoothness (CSS) values.

Within some embodiments the amplitude, strength or overall health of breathing is determined from the hand-to-hand ECG.

At least some embodiments of the present invention provide for a method for screening patients for symptoms related to a respiratory infection by monitoring the breathing and heartbeat of an individual via hand-to-hand electrocardiograph, the method comprising the steps of: a first phase of recording the heartbeat of the individual via hand-to-hand electrocardiograph (ECG) during normal breathing, a second phase of recording the heartbeat of the individual via hand-to-hand ECG during controlled or paced breathing, comparing the first and second recordings or separately analyzing the second phase to arrive at an indication of a state of function of the autonomic nervous system of an individual. Within certain embodiments a statistical analysis of the breathing data is performed in order to arrive at an index representative of state of health, such as a breathing index or vagus health index.

Within at least some embodiments of the present invention the normal breathing data and controlled or conscious breathing data is compared in order to arrive at an index indicative of the state of health of the autonomous nervous system, such as a breathing index, respiratory sinus arrythmia synchronization, cardiac sinus smoothness, respiratory sinus smoothness, vagus health index, or vagus breathing index.

In certain embodiments, a plurality of indices, such as at least two of: VBI, RSAsync and VHI, are calculated and combined into a three-dimensional matrix and there comparing the latest test results to historical values and from these results establishing a trend which forms the basis for a personalized health care monitoring and diagnostics.

At least some embodiments provide for a method further comprising the step of combining both the values of RSS and CSS into a matrix and there comparing the latest test results to historical values and from these results establishing trends for personalized health care monitoring and diagnostics.

Systems according to at least some embodiments of the present invention comprise:
A) A wearable ECG device in the form of a single lead electrocardiogram enabled smartwatch or -ring. This wearable ECG device can transfer measured data to a smartphone or direct to the cloud through wireless transfer such as WIFI or SIM-card enabled data transfer. If the device cannot communicate directly wirelessly with the below mentioned CDPSU, then the system may employ the third part mentioned in C).
B) A Central Data Processing and Data Storage Unit (CDPSU) either as cloud processing or other solution. This cloud processing enables the analytics of all collected data, storage of historical data and distribution of data to third parties such as for instance doctors or other third parties. This central part of the system also contains so-called bots which together with artificial intelligence can advise and help the user to better manage his/her health and disease.
C) A smartphone, pad, computer or other type of data transfer and user interface display device with an application software which is receiving the data from the wearable ECG device, storing the data, displaying the data and analysis results, transferring and receiving data and analytics results from the central data processing and storage unit.

A ‘Vagus ECG Test’ according to certain embodiments of the present invention consists of a normally 90 seconds long electrocardiogram test. The first 30 seconds is a base ECG recording when the person is sitting still and does normal breathing. During the next 60 seconds, the person does so-called controlled breathing where each deep inhale and exhale lasts 5 seconds.

In another embodiment the ‘Vagus ECG Test’ consists of a normally 30 seconds long electrocardiogram test. The 30 seconds ECG recording is done when the person is sitting still and does so-called paced (controlled) breathing where each deep inhale and exhale lasts 5 seconds. This breathing pattern can also be described as a ‘0.1 Hz paced breathing’.

The wearable device according to some embodiments has a display function which instructs the user of how to do the 30-90 seconds' test.

After doing this test, the wearable ECG device transmits the data via to the transfer and display device which then transmit the data to the CDPSU for analysis. The analytics results are transmitted back to the phone/device A) and C) within a short time period-normally a few seconds.

Certain systems according to the present invention compromise some of the following features:
a) Signal transfer to and from the device with Wireless Bluetooth, Zigbee or other radio transmission to either a smartphone or directly to the data cloud by wireless data transmission.
b) Signal transfer, processing and feedback in a smartphone and its dedicated app.
c) Cloud processing
d) At least some embodiments also comprise:
   a. Individualization
   b. Neurofeedback from non-invasive or invasive vagus- or other neurostimulation
   c. Feedback from autonomic nervous system interventions with pharmacology, neurostimulation or health/wellness treatments.

Disease monitoring and detection systems according to some embodiments compromise at least the following analytical methods where the system is combining these parameters to a standardized evaluation of changes to the persons immune response and hence probability for that the person to have contracted a disease:
a) The here presented novel Respiratory Sinus Arrythmia Synchronization calculation consisting of estimating each breath corresponding heart rate pattern and timing and establishing an index (RSAsync) which is expressing the degree of normality for this synchronization.
b) The here presented novel Cardiac Sinus Smoothness Index (CSS) as calculated from the smoothness of the cardiac waveform during the 0.1 Hz controlled breathing test-phase.
c) The here presented novel Respiratory Sinus Smoothness Index (RSS) as calculated from the smoothness of the systems derived respiratory waveform during the 0.1 Hz controlled breathing test-phase.
d) The heart rate QRST pattern and amplitude so that for instance amplitude decrease and/or T-wave amplitude and placement increase represent.
In addition to above analytical methods the here presented system also includes at least one of the following parameters when it establishes changes to the persons immune system and probability to have contracted a disease:
e) Heart Rate Variability (HRV) as calculated from ECG- and optical pulse measurements f) Pulse as measured both with ECG and the watch/ring optical pulse sensor
g) Blood Pressure as measured with the watch/ring optical pulse and blood pressure measurement algorithms
h) Electrodermal activity as measured with the watch/ring galvanic skin response sensor
i) Pulse pattern changes between the normal breathing and controlled breathing phase in the Vagus ECG Test.
j) Personalization with artificial intelligence systems based on the person's previous data and all user's general data.

The hand-to-hand ECG signals have many similarities to the signals from the standard 12-lead electrode placements called a VR and aVL. It is estimated that by 2022 there will be 200 million hand-to-hand ECG capable smart watches and fitness bands. Hand-to-hand ECG will hence become the 2nd most common home health diagnostics device after the thermometer.

At least some devices according to the present invention are waterproof so they can be easily disinfected between use if used for multiple users. In such multi-user applications, the device may be configured to log user name and data.

Within certain embodiments, the Vagus ECT Test disease assessment is done in the cloud and it is shown on the phone app within seconds from finishing the test.

When certain embodiments of the invention are sold and used as a wellness device, the analytics and system only inform the user of the values presented earlier and as an index called ‘Vagal Homeostasis’—without making any medical diagnostics or statements.

In embodiments of the invention sold and used as a medical device, the analytics and system make medical diagnostics and advices the user of possible ways and methods to treat the detected disease. Certain embodiments also incorporate the option for medical consulting, at-home health monitor and general health coaching.

At least some systems enable automatic or separately instructed sharing of data, measurements or analytics results to the users defined medical professional or other party.

The here presented infection and/or other disease risk assessment combined with traditional screening methods such as measuring temperature or observing external signs such as coughing, provides a better and more comprehensive system than what is now used worldwide.

At least some embodiments of the present invention find use in the following areas:
1) Detecting the degree of oxygenation
2) Detecting the efficiency, volume and periodicity of breathing
3) Detecting dynamic blood pressure
4) Diagnosing and evaluating changes in the user's stress levels
5) Diagnosing the state and activity level of the user's immune system
6) Diagnosing and evaluating changes in inflammation levels
7) Diagnosing the recovery process if the user is suffering from myocarditis
8) Diagnosing the recovery process if the user is suffering from pneumonia or other respiratory inflammations
9) Diagnosing the recovery process after a stroke
10) Diagnosing and monitoring the recovery process after respiratory-cardiac virus based infections such as Covid-19.
11) Diagnosing when the risk for an asthma attack is increasing
12) Diagnosing and evaluating changes in the user's fatigue and chronic fatigue
13) Diagnosing and evaluating changes in the user's autoimmune diseases
14) Diagnosing and evaluating changes in the user's gut diseases such as IBD and Crohns disease
15) Screening for psychiatric diseases
16) Early detection and evaluating changes in the user's asthma attacks
17) Early detection and evaluating changes in the user's anxiety attacks
18) Early detection and evaluating changes in the user's rage outbursts
19) Early detection and evaluating changes in the user's fear response
20) Evaluation of the user's autonomic nervous system reactions to different types of music
21) Evaluation of the effects from alcohol-, drug- or other types of abuses
22) Detection of harmful sinus arrythmias
23) Providing neurofeedback for vagus nerve stimulation and neuromodulation devices and systems
24) Fit for duty tests of professional pilots and ship captains, for example ensuring they have received the required amount of rest or are not in an altered state.
25) Evaluation of the user's health and fitness levels for sports and training purposes.

At least some embodiments of the present invention are configured to:
determine at least one of a level and state of ‘vagal homeostasis’.
determine if the user's immune system has changed from the time of previous measurements and analytics results. establish a standardized test protocol, sometimes referred to as ‘Vagus ECG Main Test’, consisting of 30 seconds base breathing and then a 60 second controlled breathing period with 5 seconds inhale and 5 seconds exhale in each breathing cycle.
establish a standardized quick test protocol, sometimes referred to as ‘Vagus ECG Test’, consisting of 30 second controlled breathing period with 5 seconds inhale and 5 seconds exhale in each breathing cycle.
employ a novel and informative breathing analytics parameter which is called Vagal Breathing Index (VBI) as measurement of the breathing when calculated from the QRS amplitude changes during the controlled 30 second breathing phase of the ‘Vagus ECG Test’.
employ a novel and informative analytics parameter which is called Respiratory Sinus Arrhythmia Synchronization (RSAsync) as the measurement of the cardiac/respiratory synchronization. This value is at least partially derived from the ECG measurement obtained during the ‘Vagus ECG Test’.
calculate a Vagal Homeostasis Index (VHI), for example, a VHI may be represented by a value between 1-100 where 100 is considered as excellent vagal homeostasis and 1 is very bad or very low level of vagal homeostasis.
calculate a Respiratory Sinus Smoothness Index (RSS) which may be represented as a numerical value between 1-100 where 100 is considered as excellent vagal respiratory health and 1 is very bad or very low level of respiratory system health.
calculate a Cardiac Sinus Smoothness Index (CSS), for example a CSS which is between 1-100 where 100 is considered as excellent cardiac health and 1 is very bad or very low level of cardiac system health.
determine a five-level homeostasis rating which is an indicator of the measured person's homeostasis level and immune system activity levels and they are presented to the user as a) very high, b) high, c) moderate, d) low or e) very low or other suitable wordings or sound expressions from the devices.

In at least some embodiments the Vagal Homeostasis Index (VHI) and the disease risk rating system is established as an individualized combination of at least the following system measured values: Respiratory Sinus Arrythmia (RSA), Respiratory Sinus Arrhythmia Synchronization (RSAsync), Respiratory Sinus Smoothness (RSS), Cardiac Sinus Smoothness (CSS), Heart rate patterns and amplitude and Heart Rate Variability (HRV) In addition to using the previously stated values for establishing the disease risk rating—certain systems and methods use one or more of the following values: Respiratory sinus arrhythmia (RSA), Blood Pressure (BP) as measured with the device sensor, respiratory sinus arrhythmia synchronization (RSAsync), the heart rate QRST peak pattern and amplitude, heart rate Variability (HRV), Heart Rate (HR) as calculated either from the ECG or optical heart rate sensors, maximum heart rate during the Vagus ECG Test (HRmax), minimum Heart Rate during the Vagus ECG Test (HRmin), electrodermal activity as measured by the device galvanic skin response sensor, the strength and pattern of breathing as expressed with the inventions Vagus Breathing Index (VBI) and calculated from the Vagus ECG test, artificial intelligence systems based pattern recognition of the ECG patterns during the Vagus ECG Test and other bio signals measured by the wearable ECG watch sensor such as longer term optical heart rate variation, blood pressure, blood oxygenation, activity and sleep.

According to certain embodiments, the immune system activity levels and risk assessments are uniquely determined from the Vagal Homeostasis estimation and individualized additional combinations of the following: Respiratory Sinus Arrhythmia RSA), Respiratory Sinus Arrhythmia Synchronization (RSAsync), Heart Rate Variability (HRV), Heart Rate (HR), Maximum Heart Rate during the Vagus ECG Test (HRmax), minimum Heart Rate during the Vagus ECG Test (HRmin), Vagus Breathing Index as calculated from the Vagus ECG test (VBI), pattern recognition of the ECG patterns during the Vagus ECG Test, other bio signals measured by the wearable ECG watch sensor such as longer term optical heart rate variation, blood pressure, blood oxygenation, activity, sleep, self-reported historical information and artificial intelligence evaluations of the persons individual immune system profile and its change-patterns.

Certain embodiments use historical data and feedback from the user together with artificial intelligence and self-learning algorithms to refine and improve the analytical values, diagnostics results, conclusions and healthcare advices to improve the persons immune system activity levels and vagal homeostasis.

Certain embodiments of the present invention can determine from the immune system activity levels if the person has been infected by a bacterial or viral respiratory disease before the person has visible signs of the disease such as fever or coughing.

At least some embodiments provide a method to screen larger groups of people for respiratory—or other types of bacterial and viral diseases before the screened person has visible signs of any disease. The results can be presented as a ‘disease warning’ when vagal homeostasis, immune levels and analytics results are typical for the said disease and person type. This screening process also take into consideration such as age, sex, length and weight. The tested persons screening results is presented wirelessly to the measuring person and the relevant on- or off-site authority monitoring of the screening.

The disease risk-assessment and screening process devices and methods provided by some embodiments can additionally also use a device camera for detection parameters such as skin color, temperature and other visible features detectable with artificial intelligence together with camera vision. This risk-assessment can be used for critical work tasks and jobs as a screening method to also to detect other types of homeostasis imbalances and immune stress.

Certain devices and systems according to embodiments of the present invention can be mental stress levels, depression, anxiety, chronic neurological low-grade inflammations or digestive and gut imbalances. Some devices and systems can detect and evaluate the degree and level of the tested persons ‘Post Traumatic Stress Disorder (PTSD). A portion of devices and systems provide a new diagnostics and analytics method with user feedback capability for evaluating the effect of pharmaceutical medication dosing and it provides a means to establish personalized medication.

At least some devices and systems according to embodiments of the present invention provide a new analytics and evaluation method for pharmaceutical medication trials. Certain devices and system provide a new type of analytics and evaluation models for Alzheimer and dementia when they occur as a result of neurological inflammations. Still some devices and methods provide a new method to provide feedback for personalized pharmaceutical medication.

Some devices and systems according to embodiments of the present invention enable long-term health tracking, longer-term data collection and big data machine learning and artificial methods for the system to can detect if the person is becoming sick or if the low vagal homeostasis is due to mental issues, neurological low-grade inflammation or issues connected with the gut and digestions. With personalized long-term health tracking, longer-term data collection and big data machine learning and artificial methods the system can detect if the person long-term changes in vagal homeostasis is most likely to occur due cancer-like tumors.

Certain analytics systems according to embodiments of the present invention consists partly of machine learning algorithms which can improve the differentiation between these previously mentioned causes of ‘vagal homeostasis’ changes.

Risk-assessments according to embodiments of the present invention can be used to detect high stress, high degrees of depression, potentials for suicidal thoughts and/or other mental states which seriously can harm the tested person's life or work capability.

Embodiments of the present invention may be used: for cardiac-respiratory viral diseases such as the Coronavirus COVID-19; to detect heart muscle- and lung muscle inflammations; to monitor post stroke health progress and treatments; to detect changes in the persons Cholinergic Immune response; to evaluate and monitor the effects on brain- and neurostimulation devices and methods.

The graphical representation of the Vagus ECG Test breathing and pulse changes, as provided by certain embodiments of the present invention, for example by the central HEART/BREATH button on the Vagus Watch Apps, is a very valuable tool for interpretation of a Vagus ECG Test result. It provides an indication as to how the heart and breathing are synchronizing during a controlled breathing test. For example, a test comprising 5 seconds inhale and 5 seconds exhale as controlled breathing which is done during the second (60 sec) part of at least some tests according to the present invention. Within these indications of synchronization, it is visually easy to notice if the test person cannot create ‘calm’ waves for both the heart rate and breathing. If the test person cannot ‘create’ this very important physiological reaction through breathing, then there probably is some degree of negative health issues, for example within the heart-, lungs or autonomic nervous system. These health issues are different for different persons and tests according to the present invention may provide for a warning and notification for the user to seek professional medical help if the test results are, for example, persistent over several days, found in more than 6 tests and no other natural explanation can be found for the discrepancies.

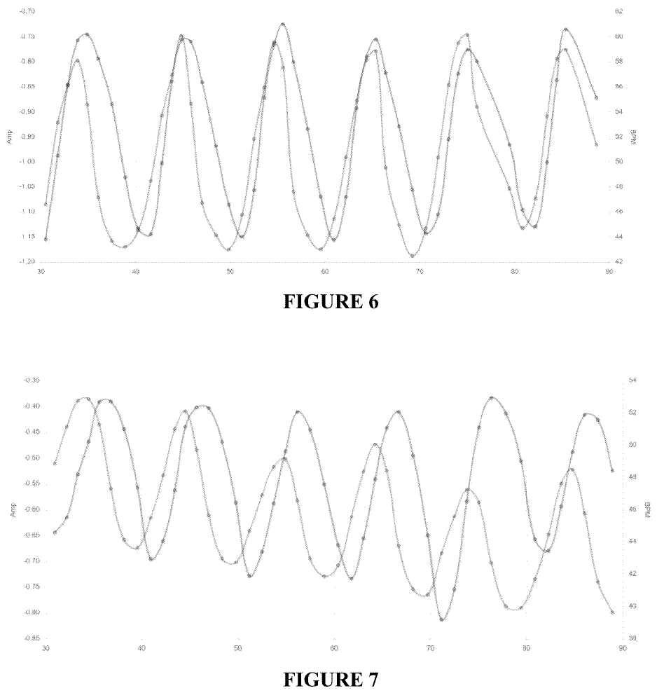

Within the matrix of FIG. 5, there is provided examples of visual interpretations according to the present invention. The y axis represents breathing and x axis heart rate. These visual interpretations can be made via, for example, software analysis of the results performed in a variety of fashions. For example, the test results may be analyzed on the measurement device, for example a watch, itself. In other examples, the analysis can be performed on a remote server.

The hypothesized health problems illustrated within FIG. 5 are based on case-experiences from test persons using the Vagus ECG watch which may implement embodiments of the present invention.

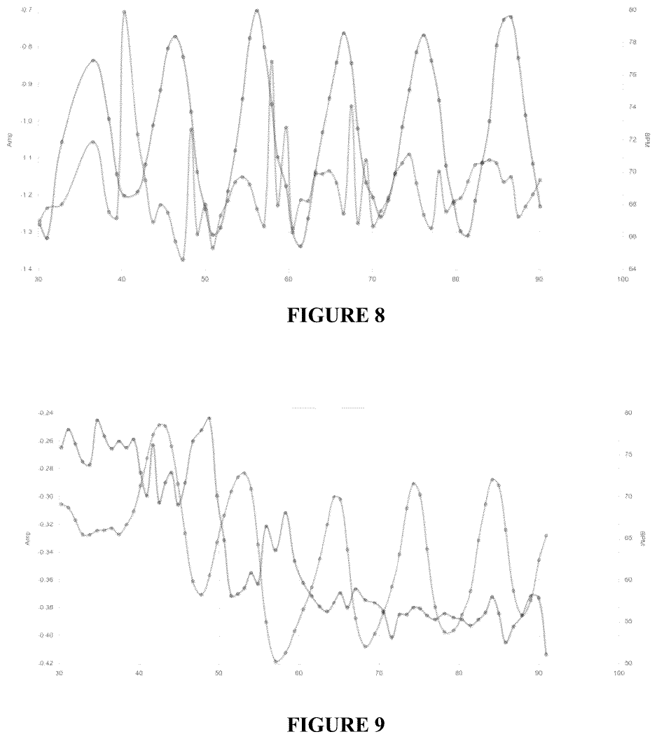

FIG. 6 shows results of a test according to an embodiment of the present invention showing two wave. The two waves are the pulse wave, having the first peak of lower amplitude, and the breathing wave having the first peak of a higher amplitude than the pulse wave. FIG. 6 shows an individual with good autonomic control, high vagal tone and is generally indicative of a healthy individual.

The individual was a 61 year old male in good health and the test was taken at mid-day. The heart rate (HR) average 51 beats per minutes, the maximum heart rate HRmax was 61 beats per minute with a minimum heart rate, HRmin of 42 beats per minute. Heart Rate Variability, HRV (SDNN) was 173, Respiratory Sinus Arrhythmia Index (RSA, 0-100): 94; Respiratory Sinus Arrhythmia Synchronization Index (RSAsync, 0-100): 90; RSS: 95, CSS: 87; Vagus Health Index (VHI, 0-100): 92 and Respiration Index (Breathing, 0-100): 48.

Generally, the individual which provided results shown in FIG. 6 has done daily vagus ECG tests for months. The above results show good high heart rate variability, higher than his average test results. Normal HRmin—in line with average good resting heart rate. Good RSA and RSAsync. Breathing normal for said test person. The pulse wave is a bit ‘late’ compared to breathing. For optimal oxygenation the pulse should rise slightly earlier. Overall a very good test result.

FIG. 7 also shows good autonomic control, High Vagal Tone, and a generally healthy individual. Within FIG. 7, the pulse wave starts at a value of around −0.5 and the breathing wave starts at a value of around −0.65. FIG. 7 was derived from a female of 40 years old who is a yogi, in good health and was conducted in the morning. The following results were derived:
Pulse, beats per minute (HR, BPM): Avg 46, Hrmax 51, Hrmin 40 Heart Rate Variability, HRV (SDNN): 111

### Respiratory Sinus Arrhythmia Index (RSA, 0-100): 95

### Respiratory Sinus Arrhythmia Synchronization Index (RSAsync, 0-100): 85

### RSS: 88, CSS: 92

### Vagus Health Index (VHI, 0-100): 90

### Respiration Index (Breathing, 0-100): 81

The person in the example of FIG. 7 has done daily vagus ECG tests for months. Good high heart rate variability, higher than her average test results. Very strong breathing. Normal HRmin (early morning test), in line with her average good resting heart rate as a practicing yogi. Very good RSA and RSAsync. The pulse wave is almost perfect. She often has a declining pulse during the controlled breathing test period. Overall a very good test result, in line with her average test results.

FIG. 8 illustrates results indicating: Good respiratory effect but low cardiac synchronicity. Low Vagal Tone. The discrepancy illustrated is probably due to a cardiac health problem. The example was provided by a male, 68 yrs who was infected with COVID-19 about 3 months before this test. Within FIG. 8 the heart wave has the lower amplitude first peak and the breathing wave has the higher. The particulars of the test are as follows:
Pulse, Heart Rate (HR, beats per minute, BPM): Avg 69, Hrmax 82, Hrmin 61 Heart Rate Variability, HRV (SDNN): 61

### Respiratory Sinus Arrhythmia Index (RSA, 0-100): 0

### Respiratory Sinus Arrhythmia Synchronization Index (RSAsync, 0-100): 68

RSS: 90, CSS: 11

### Vagus Health Index (VHI, 0-100): 34

### Respiration Index (Breathing, 0-100): 66

FIG. 8 shows good high heart rate variability, higher than his average test results. Normal Pulse, HR and HRmax. HRmin—higher than the persons average resting heart rate. Very bad RSA and RSAsync. Breathing normal for said test person. The pulse wave is not synchronizing with breathing. Several quick beats. We recommend to visit a cardiologist if these test results are consistent for several days during the morning and evening Vagus ECG Tests.

FIG. 9 illustrates results showing good cardiac vagal reacting to breathing but very low respiratory amplitude change and synchronicity to breathing. Low Vagal Tone. The discrepancy is probably due to a respiratory inflammation, general inflammations or autonomic nervous system related health problems. Within FIG. 9 the heart wave starts at the lower value and the breathing wave at the higher. This example was provides by a male of 45 years of age and had the following particulars:
Pulse, Heart Rate (HR, beats per minute, BPM): Avg 65, Hrmax 74, Hrmin 52 Heart Rate Variability, HRV (SDNN): 127

### Respiratory Sinus Arrhythmia Index (RSA, 0-100): 26

### Respiratory Sinus Arrhythmia Synchronization Index (RSAsync, 0-100): 74

### RSS: 25, CSS: 82

### Vagus Health Index (VHI, 0-100): 50

### Respiration Index (Breathing, 0-100): 22

FIG. 9 shows high heart rate variability. Normal Pulse and HRmax. The resting heart rate, HRmin is within the normal range. Low RSA. The controlled breathing process were done normally during the tests (the test person has done several weeks of tests before the respiratory synchronicity decline). The lack of respiratory effect may be attributed to severe stress, lack of muscular control, cardiac and/or lung tissue muscle infections or genetic disposition. Such a result could result in a recommendation to visit a doctor for inflammation marker blood tests if the Vagus ECG Test continues to be consistently outside of the person's normal values during several days of morning and evening Vagus ECG Tests.

FIG. 10 shows no visible correlations between heart rate variations and breathing. Neither correlates to the 5 sec inhale/ 5 sec exhale performed by the test person. Pulse and breathing amplitude are not in synchronicity with each other. Low Vagal Tone. Possible vagus nerve- or other health problems. Within FIG. 10 the heart wave initially starts at the higher value. The results here were provided by a male of 57 years and had the following particulars.
Pulse, Heart Rate (HR, beats per minute, BPM): Avg 74, Hrmax 86, Hrmin 65 Heart Rate Variability, HRV (SDNN): 84

### Respiratory Sinus Arrhythmia Index (RSA, 0-100): 46

### Respiratory Sinus Arrhythmia Synchronization Index (RSAsync, 0-100): 63

### RSS: 60, CSS: 41

### Vagus Health Index (VHI, 0-100): 55

### Respiration Index (Breathing, 0-100): 50

FIG. 10 shows high heart rate variability. Normal Pulse and HRmax. The HRmin is higher than the persons average resting heart rate. Low RSA and RSAsync. Breathing value is low but normal for said test person. The pulse wave is not synchronizing with breathing or pulse variations. Despite that the person is breathing according to the Vagus ECG Test periods, the QRS amplitude breathing effect is irregular. We recommend visiting a doctor if these test results continue for several days during the morning and evening Vagus ECG Tests.

FIG. 11 shows results derived from a 30 year old male, showing good Health, No controlled breathing, normal breathing during the Vagus ECG Test final phase. Within FIG. 11 the heart wave begins at a higher value than the breathing wave. The particulars of the test of FIG. 11 are as follows:
Pulse, Heart Rate (HR, beats per minute, BPM): Avg 67, Hrmax 72, Hrmin 62 Heart Rate Variability, HRV (SDNN): 44

### Respiratory Sinus Arrhythmia Index (RSA, 0-100): 50

### Respiratory Sinus Arrhythmia Synchronization Index (RSAsync, 0-100): 70

### RSS: 58, CSS: 43

### Vagus Health Index (VHI, 0-100): 60

### Respiration Index (Breathing, 0-100): 15

Within FIG. 11 the heart rate is having a longer periodicity variation which is correlating with some delay to the cardiac QRS amplitude changes. These changes are not caused by the physiological Brody effect, but may be fluctuating due to autonomic nervous system homeostasis adjustments. The interval and amplitude of these homeostasis adjustments are individual and depending on issues such as environment, digestion, sleep, stress etc. and are reflecting natural variations whereby the body is working to achieve optimum homeostasis.

FIG. 12 shows results derived from an 80 year old female diagnosed with heart valve dysfunction but otherwise in good health. The heart wave starts at a higher amplitude than the breathing wave. The particulars of the test are:
Pulse, Heart Rate (HR, beats per minute, BPM): Avg 66, Hrmax 77, Hrmin 55 Heart Rate Variability, HRV (SDNN): 110

### Respiratory Sinus Arrhythmia Index (RSA, 0-100): 0

### Respiratory Sinus Arrhythmia Synchronization Index (RSAsync, 0-100): 53

### RSS: 60, CSS: 3

### Vagus Health Index (VHI, 0-100): 27

### Respiration Index (Breathing, 0-100): 90

Within test of FIG. 12, breathing was good but the heart rate shows no synchronization to breathing. Very low vagal tone. A cardiologist is regularly monitoring the test persons ECG.

As presented above, the results of a vagus ECG test according to at least some examples of the present invention may provide for many indications of a person's current condition. In at least some instances the test may provide an indication of inflammation, for example inflammation or other symptoms common to a COVID-19 infection. Further, certain embodiments provide for an analysis of patients after a COVID-19 infection.

Certain embodiments provide for an indication that further test may be needed. For example, Vagus ECG watches may be provided to a variety of patients for continuous monitoring which may be performed independently by the patients. Such further test may include, for example, Magnetic Resonance Imaging (MRI) tests.

In at least some embodiments, a user is provided an indication that they should go for a walk or otherwise experience light exercise between tests. Such testing can be useful to provide further details as to the user's current condition as the two tests can be compared.

In at least some embodiments of the present invention, the devices and methods are used for health monitoring, medical diagnostics and personalization of health care.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

1. A smartwatch for determining a state or activity level of the autonomic nervous system, the smartwatch comprising:
at least a pair of electrocardiograms (ECG) measurement electrode pads configured to perform hand-to-hand ECG measurements, and
at least one processing core and at least one memory including computer program code, the at least one memory and the computer program code being configured to, with the at least one processing core, cause the smartwatch to:
monitor the pair of ECG measurement pads during a first phase to arrive at a first reading of hand-to-hand ECG,
monitor the pair of ECG measurement pads during a second period, while providing instructions to perform controlled breathing, to arrive at a second reading of hand-to-hand ECG,
analyze the of hand-to-hand ECG readings during the first and second phase to arrive at an indications of Respiratory Sinus Arrhythmia Synchronization calculated as a measure of cardiac and respiratory synchronization, wherein the first and second readings are compared in order to arrive at the indication.

2. The smartwatch of claim 1, wherein the smartwatch is further configured to analyze the first and second readings of hand-to-hand ECG to arrive at indications of at least one of the following:
a minimum, maximum and average pulse,
a size of pulse changes during the test,
a smoothness of pulse changes,
respiratory rate changes during the test,
a depth of respiration, and
a smoothness of respiration.

3. (canceled)

4. (canceled)

5. The smartwatch of claim 1, wherein the smartwatch is further configured to provide the instructions to perform controlled breathing such that the user is prompted to breathe at a frequency of 0.1 Hz.

6. The smartwatch of claim 1, wherein the first period is at least 30 seconds and the second period is at least 60 seconds.

7. (canceled)

8. The smartwatch of claim 1, wherein the smartwatch further comprises a display which displays the instructions regarding breathing.

9. (canceled)

10. The smartwatch of claim 1, wherein the smartwatch is configured to prompt a user in coordination with a mobile phone.

11. A method of determining a state of health for an individual by monitoring the individual's breathing via hand-to-hand electrocardiograph on a smartwatch, the method comprising the steps of:
a first phase of recording the individual's heartbeat via hand-to-hand electrocardiogram a smartwatch during normal breathing,
a second phase of recording the individual's heartbeat via hand-to-hand ECG on said smartwatch during paced or controlled breathing, and
comparing data from the first and second phase of recording to arrive at an indication of a state of function of the individual's autonomic nervous system,
wherein the indication comprises a Respiratory Sinus Arrhythmia Synchronization calculated as a measure of cardiac and respiratory synchronization, and wherein the first phase lasts at least 30 seconds and the second phase lasts at least 30 seconds.

12. The method of claim 11, wherein the indication further comprises a Respiratory Sinus Smoothness Index calculated as a degree of error between a breathing curve derived from data from the second phase of recording and an optimal normal or asymmetric sinus curve most fitted to data of the breathing curve.

13. The method of claim 11, wherein the indication further comprises a Cardiac Sinus Smoothness calculated as a degree of error between a cardiac curve derived from data from the second phase of recording and an optimal normal or asymmetric sinus curve most fitted to data of the cardiac curve.

14. The method of claim 11, wherein the indication further comprises a Vagus Breathing Index which is calculated from QRS amplitude changes.

15. (canceled)

16. The method of claim 11, wherein the amplitude, strength or overall health of breathing is determined from the hand-to-hand ECG.

17. A method for screening patients for symptoms related to a respiratory infection by monitoring the breathing and heartbeat of an individual via hand-to-hand electrocardiogram a smartwatch, the method comprising the steps of:
a first phase of recording the heartbeat of the individual via hand-to-hand electrocardiogram a smartwatch during normal breathing,
a second phase of recording the heartbeat of the individual via hand-to-hand ECG on the smartwatch during controlled or paced breathing, and
comparing the first and second recordings or separately analyzing the second phase to arrive at an indication of a state of function of the autonomic nervous system of an individual,
wherein the first phase lasts at least 30 seconds and the second phase lasts at least 30 seconds.

18. The method of claim 17, wherein a statistical analysis of the breathing data is performed in order to arrive at an vagus health index representative of state of health.

19. The method of claim 17, wherein the normal breathing data and controlled or conscious breathing data is compared in order to arrive at a vagus health index indicative of the state of health of the autonomous nervous system.

20. The method of claim 13, wherein a plurality of indices, such as at least two of: VBI, RSAsync and VHI, are calculated and combined into a three-dimensional matrix and there compared to historical values, and from these results a trend is established for personalized health care monitoring and diagnostics.

21. The method of claim 14, further comprising the step of combining both the values of RSS and CSS into a matrix and there comparing the latest test results to historical values and from these results establishing trends for personalized health care monitoring and diagnostics.

22. The method of claim 17, wherein the combined evaluation of breathing and cardiac-health determine together a general health diagnostic from the paced breathing hand-to-hand ECG.

23. (canceled)
